(19) United States
(12) Patent Application Publication
(10) Pub. No.: US 2004/0230119 A1
(43) Pub. Date: Nov. 18, 2004

(54) Echogenic stent

(76) Inventors: John R. Brustad, Dana Point, CA (US); Raffi S. Pinedjian, Fountain Valley, CA (US)

Correspondence Address: APPLIED MEDICAL RESOUCES CORPORATION, 22872 Avenida Empresa, Rancho Santa Margarita, CA, 92688, US

(21) Appl. No.: 10/844,231

(22) Filed: May 11, 2004

Related U.S. Application Data
(60) Provisional application No. 60/471,092, filed on May 15, 2003.

(51) Int. Cl. A61B 8/00

(52) U.S. Cl. 600/442

(57) ABSTRACT

An echogenic medical device such as a stent for insertion into a biological tissue or vessel comprising an elongate tube and having at least one lumen extending substantially along a longitudinal axis. The elongate tube comprises a material having an acoustic impedance different from the acoustic impedance of the biological tissue or vessel of a patient body such that ultrasonic imaging of the tube inside the patient's body may be achieved. The elongate tube may comprise a plastic material such as polyethylene or any formable, pliable material which may be molded and/or extruded to a variety of shapes depending upon a specific application. The invention improves ultrasonic imaging by entrapping air in a lumen of the stent, which may have any cross-sectional shape and which may be sealed anywhere along the longitudinal axis. The stent may include curled ends for positioning the stent in a body vessel, a plurality of holes for draining fluid in a body tissue, and additional lumen to entrap air and to further enhance ultrasonic imaging of the stent. The coils of the curled ends may also be tightly spaced together to provide ultrasound reflection. In another aspect of the invention, a material such as a foaming agent may be added during the extrusion process to cause the formation of CO₂ gas to form in the material as it is being extruded. In yet another aspect of the invention, a mesh stent may be used to advantage either by modifying the individual elements to be more echogenic or by adjusting the braid configuration to be more reflective.

This is a non-provisional application claiming the priority of provisional application Ser. No. 60/471,092, filed on May 15, 2003, entitled “Echogenic Stent,” which is fully incorporated herein by reference.

### BACKGROUND OF THE INVENTION

1. Field of the Invention

This invention generally relates to medical devices and, in particular, to echogenic stents that may be inserted into body cavities and conduits and that may be tracked using ultrasonic imaging devices.

2. Discussion of the Relevant Art

Ultrasonic imaging is commonly used in the medical field to provide imaging of physiological structures and tissues such as organs and vessels. Ultrasonic imaging has been used to track small medical devices such as needles and catheters. These small devices, however, do not provide strong ultrasound images because of their limited reflective surfaces. In the area of urology, for example, physicians still rely on fluoroscopy to place ureteral stents in patients. This procedure typically requires going to a hospital equipped with fluoroscopic equipment since most physicians do not have such equipment in their offices. As such, it would be advantageous and more cost effective if stents, among other medical devices, could be placed and tracked using ultrasonic imaging equipment during office visits instead of using fluoroscopic equipment in hospital operating rooms since many urologists already have ultrasonic imaging equipment in their offices.

Many have attempted to enhance ultrasonic imaging of smaller medical devices by modifying the reflective surface characteristics of these devices. For example, grooves have been cut in the tips of needles to provide a defraction grating on the surface and thereby improve the ultrasonic reflection of the needles. In particular, the spaced grooves can provide constructive interference of ultrasound reflections and, consequently, yield maximum reflection to the ultrasonic imaging equipment. Others have attempted to coat, embed and/or mix sound reflective particles in the material of the devices. This method relies on the gross impedance differences or mismatches between the particles and the tissue or vessel of a patient body. The above and other coating methods have suffered from many problems including difficulties in applying coatings to different substrates, excessive coating thickness, and the complexity of modifying the reflective surface of a device. Accordingly, although the current systems have made some progress, there remains a need for improved medical devices that are simple in design and easy to manufacture. More specifically, these devices should be easy to place and track during an office visit without requiring the use of hospital's fluoroscopic equipment.

### SUMMARY OF THE INVENTION

The present invention is directed to an echogenic medical device such as a stent having a high degree of ultrasonic reflection. The echogenic stent comprises an elongate tube designed to be advanced to a desired location of a biological tissue or vessel of a patient. The tube has a lumen extending substantially along a longitudinal axis, wherein at least a portion of the lumen is sealed to entrap air to enhance ultrasonic imaging. The elongate tube comprises a material having an acoustic impedance different from the acoustic impedance of the biological tissue or vessel of the patient and, as a result, ultrasonic imaging of the tube inside the patient's body is further enhanced. The stent may further comprise a second lumen for draining fluid from a body tissue or cavity. The elongate tube may comprise a plastic material such as polyethylene or any formable, pliable material that may be molded and/or extruded to a variety of shapes depending upon a specific application. The lumen may have any cross-sectional shape and may be sealed anywhere along the longitudinal axis. A structure such as a coiled spring may also be formed into the elongate tube to facilitate kink resistance and ultrasonic reflection. It is appreciated that the spacing between the coils may vary along the tube to adjust the amount of ultrasonic reflection.

The echogenic stent may further include curled ends for positioning the stent in a body vessel or passageway such as the ureter, and a plurality of holes or ports operably connected to the lumen for draining fluid in the body tissue or cavity such as the kidney or bladder. The stent may have more than one lumen for entrapping air and further enhancing ultrasonic imaging of the stent. The air-entrapping lumen has a diameter of about 1 mm, but it should be understood that ultrasound wavelengths vary considerably depending on frequency and tissue type and, thus, the air-entrapping lumen may vary accordingly depending upon a specific application. It is further appreciated that the coils of the curled ends may also be used to provide ultrasound reflection. The coils may be spaced apart to provide proper reflection of sound waves. It is further appreciated that the spacing between the coils of the curled ends may also vary depending on the application and frequency of the ultrasound.

In another aspect of the invention, a stent may be extruded having a lumen designed to be advanced to a desired location and including a plurality of porous particles or air bubbles. In particular, a material such as a foaming agent may be added during the extrusion process to cause the formation of CO₂ gas to form in the material as it is being extruded. The formation of CO₂ gas does not alter appreciably the shape of the extrusion or the properties of the finished stent. Instead, the CO₂ gas provides stronger reflection to ultrasound than the stent material alone. In yet another aspect of the invention, a mesh stent having a lumen designed to be advanced to a desired location may be used to advantage either by modifying the individual elements to be more echogenic or by adjusting the braid configuration to be more reflective.

In another aspect of the invention, a process of manufacturing the echogenic stent includes forming an elongate tube from a formable material having a lumen, sealing at least a portion of the lumen to entrap air to enhance ultrasonic reflection, and shaping the elongate tube to a desired shape to be placed into a biological tissue or vessel of the patient. In one aspect of the invention, the material is a thermoplastic and the forming step further comprises heating the formable material in a molten or liquid state to form the elongate tube. In another aspect, the material is a thermoset and the forming step further comprises a chemical reaction or process to form the elongate tube. It is appreciated that the thermoplastic has an acoustic impedance different from the acoustic impedance of the tissue or vessel of a patient body. The process may further comprise mixing sound reflective particles such as hard plastic, sand and/or metal particles into the material during processing to further enhance ultrasonic reflection, or the process may further comprise adding foaming agent to the material to cause formation of CO₂ gas to form in the elongate tube.

It is appreciated that the features and advantages of the invention may also be used to improve ultrasonic imaging of other smaller medical devices such as guidewires, needles, catheters, sheaths and the like.

### DESCRIPTION OF THE DRAWINGS

FIG. 1 is a perspective view of an echogenic stent in accordance with the first embodiment of the invention;

FIG. 2 is a cross-sectional view of the echogenic stent of FIG. 1;

FIG. 3 illustrates the approximate spacing between the coils of the echogenic stent of the invention;

FIG. 4 is a schematic diagram of a method of fabricating the echogenic stent of the invention;

FIG. 5 illustrates an echogenic stent of the invention inserted in a patient and tracked using an ultrasonic imaging device;

FIG. 6 illustrates an echogenic mesh stent in accordance with another embodiment of the invention;

FIG. 7 illustrates an echogenic stent having a coiled spring formed into the elongate tube in accordance with another embodiment of the invention; and

FIG. 8 illustrates an echogenic stent formed from a material mixed with sound reflective particles in accordance with another embodiment of the invention.

### DESCRIPTION OF THE INVENTION

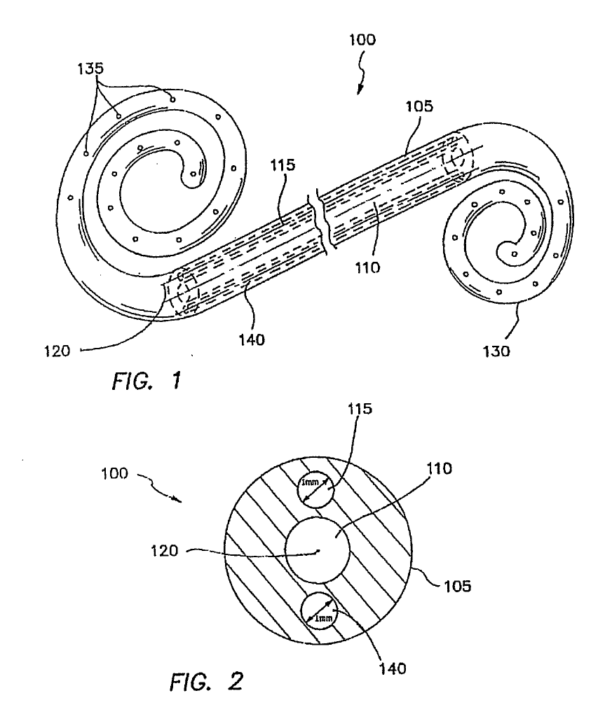

FIG. 1 illustrates an echogenic stent 100 that is used for ultrasonic imaging in accordance with the first embodiment of the invention. Echogenic stent 100 comprises an elongate tube 105 having a first lumen 110 designed to be advanced along a guidewire (not shown) to a desired location and a second lumen 115 extending substantially along longitudinal axis 120. Echogenic stent 100 may be used for insertion into a biological tissue or vessel of a patient. The elongate tube 105 comprises a material having an acoustic impedance different from the acoustic impedance of the biological tissue or vessel of the patient and, as a result, ultrasonic imaging of the tube 105 inside the patient's body may be achieved. The elongate tube 105 may comprise a plastic material such as polyethylene or any formable, pliable material which may be molded and/or extruded to a variety of shapes depending upon a specific application.

When the stent 100 is inserted into the tissue or vessel of the patient, the difference in acoustical impedance between the tissue of the patient and the material of the stent 100 is generally insufficient to provide an ultrasound reflection in response to an ultrasonic beam from an imaging equipment. A feature of the invention is to improve ultrasonic imaging of a stent by entrapping air in a lumen such as the second lumen 115 and/or additional lumen of the stent. For example, lumen 115, which may have any cross-sectional shape, may be sealed anywhere along longitudinal axis 120 to trap air thereby enhancing the ultrasonic image of stent 100. Specifically, the air trapped in closed lumen 115 provides strong reflection of ultrasound energy to increase visibility of stent 100 within a body. Preferably, with lumen 115 extending substantially along longitudinal axis 120 of stent 100, the trapped air in lumen 115 can provide for sound reflections along its entire length back to the ultrasonic receiver to generate an image.

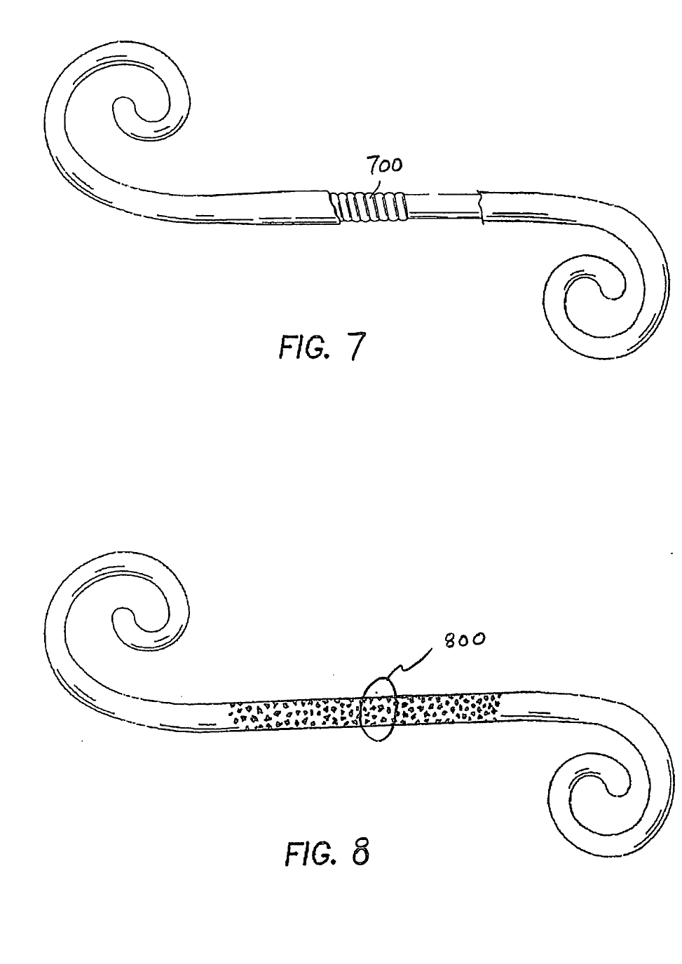

Stent 100 may further include curled ends 125 and 130 for positioning the stent in a body vessel or passageway such as the ureter, and a plurality of holes or ports 135 for draining fluid through first lumen 110 in a body tissue or cavity such as the kidney or bladder. It is appreciated that stent 100 may have more than one lumen such as third lumen 140 to entrap air and further enhance ultrasonic imaging of the stent. Referring to FIG. 7, a structure such as a coiled spring 700 can also be formed into the elongate tube to facilitate kink resistance and ultrasonic reflection. It is appreciated that spacing between the spring coils may also vary along the tube to adjust the amount of ultrasonic reflection.

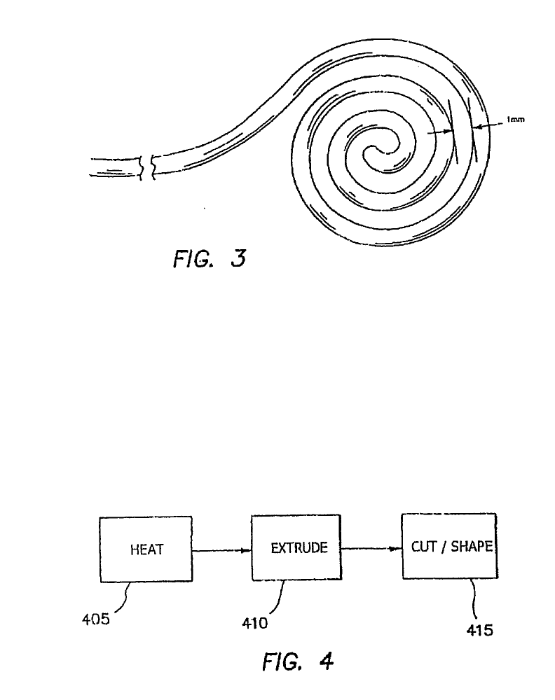

FIG. 2 illustrates a cross-sectional view of stent 100 of FIG. 1 having first lumen 110 for receiving a guidewire and second lumen 115 having a generally circular cross-section. The lumen 115 has a diameter of about 1 mm, but it should be understood that ultrasound wavelengths may vary depending on frequency and tissue type and, thus, the lumen of the stent may vary accordingly depending upon a specific application. For example, the lumen 115 may have a diameter from about 0.25 mm to about 6 mm. It is further appreciated that the coils of the curled ends 125 and 130 may also be used to provide ultrasound reflection. In particular, the coils may be spaced tightly together, for example, approximately 1 mm apart so as to provide proper reflection of sound waves as illustrated in FIG. 3. It is further appreciated that the spacing between the coils of the curled ends may also vary, e.g., from about 0.25 mm to about 6 mm depending on the application.

The process of manufacturing the echogenic stent 100 of the invention includes forming the elongate tube 105 from a formable material having a lumen, sealing at least a portion of the lumen to entrap air to enhance ultrasonic reflection, and shaping the elongate tube 105 to a desired shape to be placed into the biological tissue or vessel of the patient. In one aspect of the invention, the material is a thermoplastic and the forming step further comprises heating the formable material in a molten or liquid state to form the elongate tube 105. In another aspect, the material is a thermoset and the forming step further comprises a chemical reaction or process to form the elongate tube 105.

Referring to FIG. 4, there is shown a process of forming a thermoplastic echogenic stent of the invention. In particular, an elongate tube is heated at step 405 and then extruded at step 410 to enclose an internal lumen. The heating step 405 maintains the formable material in a molten or liquid state allowing it to be formed to the desired shape during the extruding step 410. Thereafter, the elongate tube may be cut and/or shaped at step 415 into a specified length. The elongate tube may further include drainage ports and lumens, curled and/or tapered ends, and other such processes known in the plastics and catheter industry. Prior to or during the heating step 405, the formable material may also be mixed with sound reflective particles such as hard plastic, sand, metal particles and the like, to further enhance ultrasonic imaging. FIG. 8 illustrates an exemplary echogenic stent formed from a material mixed with sound reflective particles 800.

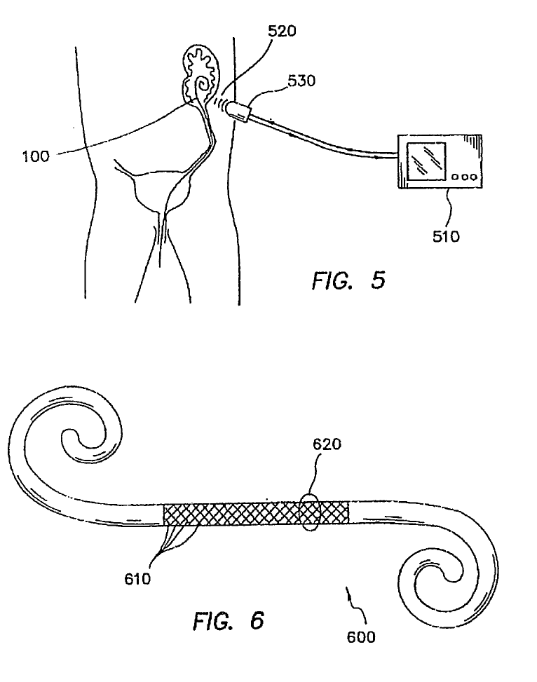

FIG. 5 illustrates the echogenic stent 100 of the invention inserted inside a patient and tracked using an ultrasonic imaging device 510. In particular, a sonic image may be produced as a physician or user guides the stent 100 through a vessel or organ of the patient. More specifically, as a sonic beam 520 is directed toward the vessel or organ of the patient with a probe 530, the stent 100 may be tracked using the stent's reflections or image from the material of the elongate tube and the enclosed lumen which are significantly different from reflections of the vessel or organ. As explained above, the acoustic impedance of stent 100 relative to its surrounding is sufficient to produce a sonic image of stent 100 in response to sonic beam 520 from imaging device 510. An advantage of the invention is that stents can be placed and tracked using ultrasound instead of fluoroscopy.

In another aspect of the invention, a stent may be extruded having a lumen designed to be advanced to a desired location and having a plurality of porous particles or air bubbles formed therein. Moreover, a material such as a foaming agent may be added during the extrusion process to cause the formation of CO₂ gas to form in the material as it is being extruded. The formation of CO₂ gas does not alter appreciably the shape of the extrusion or the properties of the finished stent. Instead, the CO₂ gas provides stronger reflection to ultrasound than the stent material alone.

In yet another aspect of the invention as illustrated in FIG. 6, a mesh stent 600 having a lumen designed to be advanced to a desired location may be used to advantage either by modifying the individual elements 610 to be more echogenic or by adjusting the braid configuration 620 to be more reflective, or a combination of the two. In the above aspects of the invention, the lumen used for advancing the guidewire may also be used for drainage.

It is appreciated that the features and advantages of the invention may also be used to improve ultrasonic imaging of other smaller medical devices such as guidewires, needles, catheters, sheaths and the like.

Although exemplary embodiments of the invention have been shown and described, many other changes, modifications and substitutions will now be apparent to those of ordinary skill in the art, without necessarily departing from the spirit and scope of this invention.

1. An echogenic stent adapted to be placed into a biological tissue or vessel of a patient, comprising:
an elongate tube having a lumen extending substantially along a longitudinal axis,
the tube being adapted to be advanced to a desired location of the biological tissue or vessel, and
at least a portion of the lumen being sealed to entrap air to enhance ultrasonic reflection.

2. The echogenic stent of claim 1, wherein the lumen comprises a first lumen and a second lumen, the first lumen being adapted to be advanced to the desired location of the biological tissue or vessel, and at least a portion of the second lumen being sealed to entrap air to enhance ultrasonic reflection.

3. The echogenic stent of claim 2, wherein the first lumen is further adapted to drain fluid from the biological tissue or vessel.

4. The echogenic stent of claim 1, wherein the elongate tube comprises a material having an acoustic impedance different from the acoustic impedance of the biological tissue or vessel.

5. The echogenic stent of claim 1, wherein the elongate tube is formed from a plastic material molded or extruded to a desired shape.

6. The echogenic stent of claim 1, further comprising a coiled spring formed into the elongate tube to facilitate kink resistance and ultrasonic reflection.

7. The echogenic stent of claim 6, wherein spacing between the spring coils may be varied to adjust the amount of ultrasonic reflection.

8. The echogenic stent of claim 1, further comprising a curled end at a distal end of the elongate tube to facilitate positioning of the stent in the biological tissue or vessel.

9. The echogenic stent of claim 8, further comprising a curled end at a proximal end of the elongate tube.

10. The echogenic stent of claim 8, wherein the curled end comprises a plurality of holes or ports operably connected to the lumen to facilitate drainage of fluid from the biological tissue or vessel.

11. The echogenic stent of claim 1, wherein the lumen has a diameter ranging from about 0.25 mm to about 6 mm.

12. The echogenic stent of claim 1, wherein the lumen has a diameter of about 1 mm.

13. The echogenic stent of claim 8, wherein the curled end comprises coils that are spaced apart to provide ultrasonic reflection.

14. The echogenic stent of claim 13, wherein the coils are spaced from about 0.25 mm to about 6 mm apart.

15. The echogenic stent of claim 13, wherein the coils are spaced about 1 mm apart.

16. The echogenic stent of claim 2, wherein each of the first and second lumen has a cross-section of any geometric shape including circular, oval, square, triangular, rectangular, pentagonal and hexagonal.

17. An medical device adapted to be placed into a biological tissue or vessel of a patient, comprising:
an elongate tube having a lumen extending along a longitudinal axis; and
a plurality of porous particles or air bubbles formed along a portion of the elongate tube,
the tube being adapted to be advanced to a desired location of the biological tissue or vessel, and
the plurality of porous particles or air bubbles being formed along the portion of the tube to enhance ultrasonic reflection.

18. The medical device of claim 17, wherein the lumen is further adapted to drain fluid from the biological tissue or vessel.

19. The medical device of claim 17, wherein the elongate tube is formed from a plastic material.

20. The medical device of claim 19, wherein the plastic material is extruded to a desired shape.

21. The medical device of claim 20, further comprising a foaming agent added during the extrusion process to cause formation of CO₂ gas to form in the elongate tube.

22. An echogenic stent adapted to be placed into a biological tissue or vessel of a patient, comprising:
an elongate tube having a lumen extending substantially along a longitudinal axis; and
a braid or mesh having a plurality of individual elements formed around a portion of the elongate tube,
the tube being adapted to be advanced to a desired location of the biological tissue or vessel, and
the braid or mesh being formed around a portion of the tube to enhance ultrasonic reflection.

23. The echogenic stent of claim 22, wherein the lumen is further adapted to drain fluid from the biological tissue or vessel.

24. The echogenic stent of claim 22, wherein the amount of ultrasonic reflection is partially determined by the configuration of the braid or mesh.

25. The echogenic stent of claim 24, wherein the amount of ultrasonic reflection is adjusted by adjusting the individual elements of the braid or mesh.

26. A process of manufacturing an echogenic stent adapted to be placed into a biological tissue or vessel of a patient, comprising:
forming an elongate tube from a formable material having a lumen;
sealing at least a portion of the lumen to entrap air to enhance ultrasonic reflection; and
shaping the elongate tube to a desired shape to be placed into the biological tissue or vessel of the patient.

27. The process of claim 26, wherein the material is a thermoplastic and the forming step further comprises heating the formable material in a molten or liquid state to form the elongate tube.

28. The process of claim 26, wherein the material is a thermoset and the forming step further comprises a chemical reaction or process to form the elongate tube.

29. The process of claim 26, wherein the material includes a thermoplastic having an acoustic impedance different from the acoustic impedance of the biological tissue or vessel.

30. The process of claim 26, wherein the elongate tube further comprises a curled end at a distal end to facilitate positioning of the stent in the biological tissue or vessel.

31. The process of claim 30, wherein the curled end comprises a plurality of holes or ports operably connected to the lumen to facilitate drainage of fluid from the biological tissue or vessel.

32. The process of claim 26, further comprising mixing sound reflective particles into the formable material.

33. The process of claim 32, wherein the sound reflective particles include at least one of hard plastic, sand and metal particles to further enhance ultrasonic reflection.

34. The process of claim 26, further comprising adding foaming agent to the material to cause formation of CO₂ gas to form in the elongate tube.

35. A medical device, comprising:
a distal section having a first flexibility and a first length; and
a proximate section having a second flexibility and a second length,
wherein a portion of at least the distal section and the proximate section includes a plurality of porous particles or air bubbles to enhance ultrasonic reflection.

36. The medical device of claim 35, wherein the device is a guidewire, a needle or a catheter.

37. A ureteral access sheath, comprising:
an elongate tube having a first lumen extending between a proximal end and a distal end; and
a handle disposed at the proximal end of the tube having a flared configuration, a first surface facing generally distally, and a second surface facing generally proximally and configured to funnel instrumentation into the lumen,
the elongate tube further comprises a second lumen, at least a portion of the second lumen being sealed to entrap air to enhance ultrasonic reflection.

38. The ureteral access sheath of claim 37, further comprising a coiled spring molded into the elongate tube to facilitate kink resistance and ultrasonic reflection.

39. The ureteral access sheath of claim 38, wherein spacing between the spring coils may be varied to adjust the amount of ultrasonic reflection.

40. The ureteral access sheath of claim 37, wherein the first surface is concave.

41. The ureteral access sheath of claim 40, wherein the second surface is convex.

42. The ureteral access sheath of claim 37, wherein the first surface is convex.

43. The ureteral access sheath of claim 42, wherein the second surface is concave.
